(19) United States
(12) Patent Application Publication
(10) Pub. No.: US 2002/0031462 A1
(43) Pub. Date: Mar. 14, 2002

(54) Apparatus for heat treatment of portions of milk and other liquid foods for, in particular, bottle-fed infants

(76) Inventor: Anders Jorn Fjendbo Jorgensen, Odense SV (DK)

Correspondence Address: JACOBSON, PRICE, HOLMAN & STERN, PROFESSIONAL LIMITED LIABILITY COMPANY, 400 Seventh Street, N.W., Washington, DC, 20004, US

(21) Appl. No.: 09/848,308

(22) Filed: May 4, 2001

(30) Foreign Application Priority Data
May 4, 2000 (DK) BA 2000 00159

(51) Int. Cl. A61L 2/00

(52) U.S. Cl. 422/307; 422/1

(57) ABSTRACT

The innovation concerns an apparatus for heat treatment of portions of milk and other liquid foods for bottle-fed infants. The heat treatment seeks by means of a pasteurization-like process to remove all risk of infection or disease transmission due to bacteria, viruses, protozoa or metazoa contained in the food concerned.

The novelty of the innovation is that the apparatus is designed for combined heat treatment of the bottle and the food portion, i.e. with the food portion placed in the bottle, enabling the feeding of the food portion concerned to take place directly following the heat treatment, at least without prior pouring or tapping off of the content into another container. The bottle is thus disinfected or sterilized together with its content so that any kind of transmission of disease during the feeding is eliminated. A specific use for the apparatus is the prevention of HIV transmission from mother to child via breast milk, as experiments have shown that heat treatment of breast milk at 62 degrees C. for approximately 30 minutes destroys the human immunodeficiency virus.

### FIELD OF APPLICATION

The innovation concerns an apparatus for heat treatment of portions of milk and other liquid foods for, in particular, bottle-fed infants. The heat treatment seeks, either by means of a pasteurisation-like process or traditional sterilisation, to remove all risk of infection or disease transmission due to bacteria, viruses, protozoa or metazoa contained in the food concerned. A specific use for the apparatus is the prevention of HIV transmission from mother to child via breast milk, as experiments have shown that heat treatment of breast milk at 62 degrees C. for approximately 30 minutes destroys the human immunodeficiency virus.

### TECHNICAL PROBLEM TO BE SOLVED

The invention seeks to provide a simple and inexpensive apparatus as stated with which breast milk, for example, can be heat-treated directly in the feeding-bottle. The apparatus is to be capable of use under primitive conditions, using a battery or solar cell panel for power supply, i.e. independently of stable mains electricity. This is one of the reasons why it has been endeavoured to achieve the lowest possible power consumption through effective thermal insulation of the apparatus. The apparatus must be simple to use, and must not require any special technical understanding on the part of users.

### DISCLOSURE AND TECHNICAL EFFECT

The novelty of the innovation is that the apparatus is designed for combined heat treatment of the bottle and the food portion, i.e. with the food portion placed in the bottle, enabling the feeding of the food portion concerned to take place directly following the heat treatment, at least without prior pouring or tapping off of the content into another container. The bottle is thus disinfected or sterilised together with its content so that any kind of transmission of disease during the feeding is eliminated. In a preferred embodiment, the apparatus comprises an upwardly open heat chamber in the form of a receptacle with a water bath for the bottle, an electric heating element positioned in conjunction with the said heat chamber, a thermally insulated external housing into which the heat chamber is fitted, a thermally insulated lid for placing over the heat chamber, and an electronic heating controller with a temperature sensor mounted in thermal contact with the heat chamber. This embodiment is distinguished by being simple, robust and easy to use. Appropriate choice of materials will permit the apparatus to be produced at a relatively low price.

The heating element and controller may conveniently be designed for 12 V d.c., making the apparatus safe to use while also enabling a number of different sources of power to be used for its operation. The apparatus can for example be operated by means of solar cells, cf. claim 4. When the apparatus is to be used in places where the mains electricity supply is unstable or non-existent (developing countries), it is in accordance with the invention advantageous that the apparatus is ready for connection to the actually available local power sources. Apart from a solar cell panel, the power supply arrangements may include, for example, a converter for the conversion of available mains voltage (for example 220 V a.c.) to 12 V d.c. operating voltage, a 12 V battery of capacity of the order of 30-60 ampere-hours with charger, and a power supply lead with a plug to fit a car cigarlighter. In this way the apparatus is able be used practically anywhere, at least wherever the necessary solar energy to operate the solar cell panel is present.

A fuller and more detailed description of the innovation and especially advantageous embodiments and particulars will now be given, with reference to the accompanying drawings.

### LIST OF DRAWINGS

FIG. 1 shows a vertical section of an apparatus according to the invention,

FIG. 2 shows a cross-section of the same (at A-A in FIG. 1),

FIG. 3 shows a side view of the apparatus, and

FIG. 4 shows a side view of the apparatus with the lid off and the bottle taken out.

### EXAMPLE EMBODIMENTS

The embodiment shown in the drawings consists in essence of a heat chamber (1), an external housing (2) with thermal insulation (3), a lid (4) with thermal insulation (5), a base (6) in the housing (2), an electronic heating controller (7) built into the base, an electric heating element (8), a temperature sensor (9) and a thermal safety cut-out (10). The base (6), which is milled out of solid plastic, is provided with a bottom-cover (11) mounted by means of screws (12). A bottle placed in the heat chamber (1) is designated ‘S’.

The heat chamber (1) is manufactured of cast aluminium, and the heating element (8), which is a standard 12 volt nozzle heating element as used in an injection-moulding machine, is fastened around the exterior of the heat chamber. The temperature sensor (9) and thermal cut-out (10) are mounted by means of screws directly on the body of the aluminium receptacle, for best possible thermal contact. The housing (2) is composed of a cylindrical outer wall (13) of plastic tubing and a collar (14) of vacuum-moulded ABS plastic. The bottom of the housing consists of the previously mentioned base (6). The parts are held together by a connecting-piece (15) and screws (16). The cavity between the outer wall (13) and the heat chamber (1) is filled with insulating mineral wool (3).

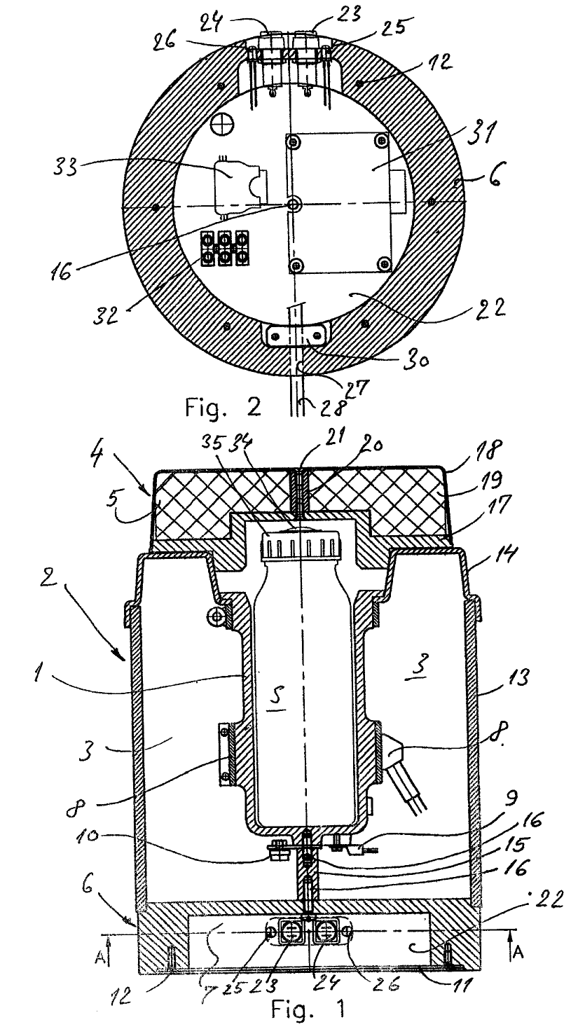

The lid (4) is composed of a bottom plate (17), a vacuum-moulded cover (18) and insulating filling (19). The parts are held together by a central connecting-piece (20) and screws (21). As previously mentioned, the base (6) contains the heating controller (7) in a milled recess (22) which is provided for the purpose and closed by means of the bottom-cover (11). The bottom-cover consists of 1.5 mm thick aluminium sheet. Two switches (23, 24), of which one is an on/off switch and the other a reset switch, are mounted on the outer surface of the base, which is suitably milled and drilled to receive them. Two LEDs (25, 26) are mounted beside the switches. The LEDs indicate the operating state of the apparatus by means of ordinary on/off function and flashing. A hole (27) is drilled through the base opposite to the switches and LEDs to admit the power supply lead (28) which terminates in a ‘cigar-lighter plug’ (not shown). FIG. 2 shows how the power lead (28) is held by a cord grip (30) inside the base. The electronic controller comprises a printed circuit board (31) on which are mounted its microprocessor etc. A terminal block is identified by (32) and a fuse by (33).

A feeding-bottle S with its teat (34) (turned downwards into the bottle) and screw cap closure (35) is lowered into a water bath in the heat chamber (1) so as to obtain the best possible thermal contact between the body of the aluminium receptacle and the bottle. The temperature is registered by the temperature sensor (9). If for any reason overheating occurs in the heat chamber with a danger of boiling dry etc., the thermal cut-out (10) will disconnect the supply of electric current to the heating element (8). The thermal cut-out is of the bimetallic type. The temperature sensor (9) is of the NTC type.

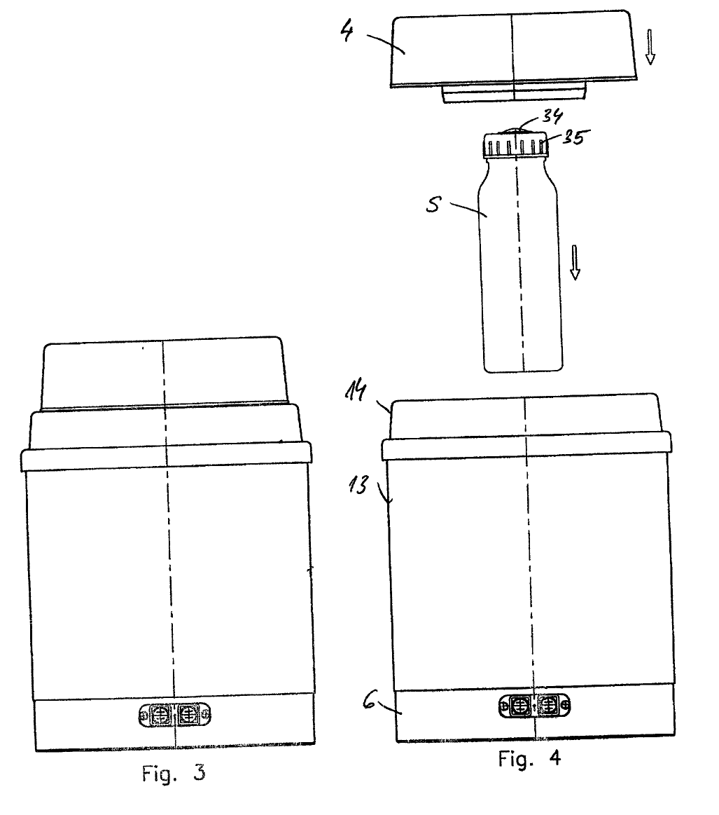

FIG. 4 shows the apparatus with the lid (4) off and the feeding-bottle (S) taken out of the heat chamber (1). FIG. 3 shows the apparatus assembled ready for use.

The invention is not limited to the embodiment shown in the drawings and described above. Other combinations of materials, other design details and other design solutions for individual apparatus components and accessories are possible within the scope of the invention, and the application of the apparatus could also be extended to other areas than stated herein.

1. Apparatus for heat treatment of portions of milk and other liquid foods for, in particular, bottle-fed infants, where the heat treatment seeks, by pasteurisation or sterilisation, to remove all risk of infection or disease transmission due to bacteria, viruses, protozoa or metazoa contained in the food concerned, characterised by the apparatus being designed for combined heat treatment of the bottle (S) and the food portion, i.e. with the food portion placed in the bottle, enabling the feeding of the food portion concerned to take place directly following the heat treatment, at least without prior pouring or tapping off of the content into another container.

2. Apparatus in accordance with claim 1 characterised by comprising an upwardly open heat chamber in the form of a receptacle (1) with water bath for a bottle (S), an electric heating element (8) positioned in conjunction with the said heat chamber (1), a thermally insulated external housing (2) into which the heat chamber is fitted, a thermally insulated lid (4) for placing over the heat chamber, and an electronic heating controller (7) with a temperature sensor (9) mounted in thermal contact with the heat chamber.

3. Apparatus in accordance with claim 1 or 2 characterised by the heating element (8) and the controller (7) being designed for 12 V d.c., and by the heating controller comprising in addition to the temperature controller a timer function for controlling processing time.

4. Apparatus in accordance with claim 1, 2 or 3 characterised by being powered by solar cells, either directly or via a battery charged by means of solar cells.

5. Apparatus in accordance with claim 4 characterised by having a power supply system that in addition to a solar cell panel comprises a converter for the conversion of available mains voltage (for example 220 V a.c.) to 12 V d.c. operating voltage, a 12 V battery of capacity of the order of 30-60 ampere-hours, and a power supply lead with a plug to fit a car cigar-lighter socket.

6. Apparatus in accordance with claim 1 characterised by the controller (7) being based on a programmable microprocessor which comprises a heating control function, a timer function, and a function for monitoring the power supplied to the heating element (8), such that the program causes the timer to be triggered when a certain lowest working current to the heating element is registered, and such that the set process temperature is kept constant for a pre-determined processing time after the registering of this state of equilibrium, whereupon completion of the heat treatment is signalled by acoustic and/or visual means.

7. Apparatus in accordance with claim 6 characterised by being equipped with a manual switch (push-button (24) or similar) whereby the timer function can be reset when a new bottle (S) is inserted into the apparatus for heat treatment.

8. Apparatus in accordance with claims 6 and 7 where its principal use is HIV transmission prevention by heat treatment of breast milk, characterised by the said microprocessor being programmed to a process temperature of 62 degrees C. and a processing time of approximately 30 minutes at that temperature.

9. Apparatus in accordance with claim 1 characterised by having built-in batteries, for example of the Ni-Cad type, and these batteries being connected in series in order to obtain a total operating voltage of approximately 12 V d.c.
